(19) United States
(12) Patent Application Publication
(10) Pub. No.: US 2014/0308377 A1
(43) Pub. Date: Oct. 16, 2014

(54) Composition for treating and preventing obesity, containing wheatgrass extract as active ingredient

(71) Applicant: GENEMATRIX INC., Gyeongi-do (KR)

(72) Inventors: Young-Mi Lee, Iksan-si (KR); Dae-Ki Kim, Jeonju-Si (KR); Sun-Hee Lee, Anyang-Si (KR)

(73) Assignee: GENEMATRIX INC., Gyeonggi-do (KR)

(21) Appl. No.: 14/353,240

(22) PCT Filed: Nov. 5, 2012
(86) PCT No.: PCT/KR2012/009227

§ 371 Date: Apr. 21, 2014

(30) Foreign Application Priority Data
Nov. 10, 2011 (KR) 10-2011-0116778

(51) Int. Cl. A61K 36/899; A23L 1/30

(52) U.S. Cl. CPC A61K 36/899; A23L 1/3002
USPC 424/750

(57) ABSTRACT

The present invention is related to a pharmaceutical composition and a dietary supplement for treating and preventing obesity, containing a wheatgrass extract as an active ingredient, it has been ascertained that the wheatgrass extract shows excellent anti-obesity effects such as significant inhibition of weight gain caused by a high fat diet, or lowering of neutral lipid levels in blood in a high fat diet-induced animal model. The wheatgrass extract of the present invention can be useful as a pharmaceutical composition and a dietary supplement for preventing and treating obesity.

### BACKGROUND OF THE INVENTION

1. Technical Field

The present invention is related to a composition containing a wheatgrass extract for the prevention and treatment of obesity disease, and the use thereof.

2. Background Art

The recent change in dietary life and reduced physical activity according to the economic development and Westernized food style, give rises to increasing the body fat and the number of obese people caused by unbalanced metabolism and thereby, the number of patients suffering from metabolic syndrome showing various cardiovascular disease such as diabetes, hyperlipidemia, thrombolic disorder etx has been inconsistently increased till now (Lee S J et al., The prevalence estimation of metabolic syndrome and its related factors based on data from general health medical examination., J Korean Soc Health Information Health Statistics., 33:pp. 119-133, 2008; Frohlich J et al., Old and new risk factors for atherosclerosis and development of treatment recommendations., Clin Exp Pharmacol Physiol., 29: pp. 838-842, 2002.)

Obesity caused by the complex mechanism such as genetic, environmental and social factor etc, shows the accumulated level of triglyceride in adipose tissue (Chua, S. C. Jr., Monogenic models of obesity., Behav. Genet., 27: pp. 277-284, 1997). The increased blood level of fatty acid and increased abdominal obesity in obese patients which is similar syndrome to hyperlipidemia and type 2 diabetes, give rise to vetricular hypertrophy (DeFronzo, R. A, Ferrannini, E. Insulin resistance. A multifaceted syndrome responsible for NIDDM, obesity, hypertension, dyslipidemia, and atherosclerotic cardiovascular disease, Diabetes Care., 14(3): pp. 173-194, 1991.) and the accumulated abdominal obesity is closely involved in the occurrence of insulin resistance (Tamori, Y., Kasuga, M. Obesity and insulin resistance., Nippon Rinsho., 67(2):pp. 236-244, 2009.), which may cause to detrimental life habit diseases such as hypertension, atherosclerosis, diabetes, hyperlipidemia etc.

To prevent from obesity has been reported to be important and in particular, the long-term and effective management and treatment of obesity is more important in obese patients and the treatment of obesity can be divided into a life habit correction method such as diet therapy, exercise therapy and behaviour therapy; drug treatment and surgical treatment etc (Mason E E., Methods for voluntary weight loss and control., Obes. Surg, 2: pp. 275-276, 1992.). Generally, the life habit correction method has been reported to have some limit to treat obesity and therefore, combined therapy with drug treatment is needed to satisfactory treatment. The use of drug for treating obesity has been tried from long years ago but only few approved drugs from FDA, for example, sibutramine drug inhibiting the reabsorption of norepinephrine and serotonin, orlistat drug inhibiting the secretion of lipase in pancreas and gastrointestinal system etc, have been reported due to the various severe adverse response (King D J, Devaney N., Clinical pharmacology of sibutramine hydrochloride (BTS 54524), a new antidepressant, in healthy volunteers., Br. J. Pharmacol., 26: pp. 607-611, 1988; Yanovski S Z, Yanovski J A, Obesity., N. Engl. J. Med., 346:pp. 591-602, 2002). However, the approved drugs also have not widely used since they have several limits to use due to several adverse responses (Padwal R, Li S K, Lau D C, Long-term pharmacotherapy for overweight and obesity., Int. J. Obes. Relat. Metab. Disord., 27: pp. 1437-1446, 2003; Thearle M et al., Obesity and pharmacologic therapy, Endocrin. Metab. Clin. North Am, 32: pp. 1005-24, 2003).

Accordingly, the synthetic anti-obesity agent showing satisfactory efficacy with no adverse response, has not still developed till now and recently, there have been tried to develop the effective drug with little adverse response from natural resources currently (George A B et al., Medicinal strategies in the treatment of obesity., Nature., 404:pp. 672-677, 2000).

Wheatgrass belonged to Gramineae family has been reported to comprise various nutritional components, for example, protein, carbohydrate, free amino acid, vitamin, inorganic substances, chlorophyll (Nagaoka H., Treatment of Germinated Wheat to Increase Levels of GABA and IP6 Catalyzed by Endogenous Enzymes, Biotechnol Progr., 21(2):405-410, 2005.), anti-oxidants (Kulkarni S D et al., Evaluation of the antioxidant activity of wheatgrass., Phytotherapy Research., 203:pp. 218-227, 2006.) and to show various activities, for example, it reduces the transfusion requirement from the patents suffering from Thalassemia Major (Marawaha R. K. et al., Wheatgrass juice reduces transfusion requirement in patients with thalassemia major., Indian Pediatr., 41:pp. 716-720, 2004.), improves the active distal ulcerative colitis (Ben-Arye E. et al., Wheatgrass juice in the treatment of active distal ulcerative colitis., Scand J Gastroenterol., 37:pp. 444-449, 2002), induces apoptosis in human cancer cells (Bonfili L. et al., Wheat sprout extract-induced apoptosis in human cancer cells by proteasomes modulation, Biochimie, 91:pp. 1131-1144, 2009), inhibits the activity of various carcinogens such as 7,2-dimethoxybenz(a)anthracene etc (Tudek B. et al., The effect of wheat sprout extract on benzo(a)pyrene., Neoplasma., 35: pp. 515-523, 1988) and inhibit the inflammatory reaction (Watzl B., Anti-inflammatory effects of plant-based foods., Int J Vitam Nutr Res., 78: pp. 293-298, 2008).

However, there has been not reported or disclosed about the therapeutic effect of a wheatgrass extract on obesity in any of above cited literatures, the disclosures of which are incorporated herein by reference.

Therefore, the present inventors have endeavored to find the effective crude drug for treating and preventing obesity through obesity induced animal model test by high-fat feed and have found that containing a wheatgrass extract reduced the body weight, level of blood triglyceride, inhibited the increased level of blood insulin and leptin, reduced the level of total lipid and triglyceride in hepatic tissue, inhibited the lipid accumulation in hepatic tissue and reduced the weight of brown adipose tissue, therefore, it can be useful in treating and preventing obesity.

### DISCLOSURE

### Technical Problem

According to one aspect, the present invention provides a pharmaceutical composition containing a wheatgrass extract for the prevention and treatment of obesity.

The present invention also provides pharmaceutical compositions containing a wheatgrass extract for the prevention and treatment of obesity disease, together with a pharmaceutically acceptable carrier.

The present invention also provides a method for treating obesity disease in a mammal comprising administering to said mammal an effective amount of an a wheatgrass extract, together with a pharmaceutically acceptable carrier thereof.

The present invention also provides a use of a wheatgrass extract for the preparation of for manufacture of medicament employed for treating or preventing obesity disease in human or mammal.

The present invention also provides a health functional food containing a wheatgrass extract for the prevention or improvement of obesity disease as an active ingredient in an amount effective to preventing and improving the disease, together with a sitologically acceptable additive.

### Technical Solution

Accordingly, it is an object of the present invention to provide a pharmaceutical composition containing a wheatgrass extract for the prevention and treatment of obesity disease, together with a pharmaceutically acceptable carrier.

The present invention also provides a pharmaceutical composition containing a wheatgrass extract for the prevention and treatment of obesity disease.

It is another object of the present invention to provide a use of a wheatgrass extract for manufacture of medicament employed for treating or preventing obesity disease in human or mammal.

It is the other object of the present invention to provide a method for treating obesity disease in a mammal comprising administering to said mammal an effective amount of a wheatgrass extract, together with a pharmaceutically acceptable carrier thereof.

The wheatgrass disclosed herein, include the sprout of Triticum genus plants such as Triticum aestivum Lamarck, Triticum vulgare, Triticum durum, Triticum compactum and the like, having been cultivated from germination for the period from 1 week to 7 weeks, preferably, 1 week to 4 weeks.

The wheatgrass extract disclosed herein comprise the wheatgrass extract which can be obtained by extracting the sprout with distilled water, alcohols such as methanol, ethanol and the like, or the mixtures thereof, preferably, distilled water, ethanol or the mixture thereof, more preferably, 30-99% ethanol, in the present invention.

The pharmaceutical composition for treating the purposed diseases could contain about 0.01 to 95 w/w %, preferably 0.5 to 80 w/w % of the above herb composition of present invention based on the total weight of the composition.

An inventive herb composition may be prepared in accordance with the following preferred embodiment.

For the present invention, above crude drug composition can be prepared by following procedure

For example, the present invention also provide a method for preparing the inventive extract comprising the steps of; washing and drying a wheatgrass at 1^{st} step; mixing dried wheatgrass with 1 to 100-fold, preferably, 1 to 50-fold volume (v/w) of distilled water, alcohols such as methanol, ethanol and the like, or the mixtures thereof, preferably, distilled water, ethanol or the mixture thereof, more preferably, 30-99% ethanol at 2^{nd} step; extracting the solution with the extraction method by the extraction with hot water, cold water, reflux extraction, or ultra-sonication extraction, preferably, reflux extraction at the temperature ranging from 10° C.˜120° C., preferably, 80° C.˜100° C., for the period ranging from 30 mins to 7 days, preferably, 1 to 6 hours at 3^{rd} step; repeating the above-described extraction process to collect each filtrate with filtration at 4^{th} step; concentrating at the temperature ranging from 90° C.˜110° C., and drying to obtain dried wheatgrass extract of the present invention.

It is another object of the present invention to provide a process for preparing the extract of the present invention as described above for the preparation of composition effective in treating or preventing the purposed diseases.

It is still another object of the present invention to provide a pharmaceutical composition comprising an extract of wheat sprout obtained by the above described process as an active ingredient for preventing and treating obesity disease.

The inventive composition of the present invention prepared by above-described process reduced the body weight, level of blood triglyceride, inhibited the increased level of blood insulin and leptin, reduced the level of total lipid and triglyceride in hepatic tissue, inhibited the lipid accumulation in hepatic tissue and reduced the weight of brown adipose tissue in obesity induced animal model test by high-fat feed, therefore, it can be useful in treating and preventing obesity.

When the oral acute toxicity of the extract was tested, the extract had no apparent effect on mortality, clinical signs, body weight changes, and gross findings at necropsy.

The pharmaceutical composition for treating purposed diseases could contain about 0.01 to 95 w/w %, preferably 0.5 to 80 w/w % of the above crude drug composition of present invention based on the total weight of the composition.

The inventive composition may additionally comprise conventional carrier, adjuvants or diluents in accordance with a using method. It is preferable that said carrier is used as appropriate substance according to the usage and application method, but it is not limited. Appropriate diluents are listed in the written text of Remington's Pharmaceutical Science (Mack Publishing co, Easton Pa.).

Hereinafter, the following formulation methods and excipients are merely exemplary and in no way limit the invention.

The inventive composition according to the present invention can be provided as a pharmaceutical composition containing pharmaceutically acceptable carriers, adjuvants or diluents, e.g., lactose, dextrose, sucrose, sorbitol, mannitol, xylitol, erythritol, maltitol, starches, acacia rubber, alginate, gelatin, calcium phosphate, calcium silicate, cellulose, methyl cellulose, polyvinyl pyrrolidone, water, methylhydroxy benzoate, propylhydroxy benzoate, talc, magnesium stearate and mineral oil. The formulations may additionally include fillers, anti-agglutinating agents, lubricating agents, wetting agents, flavoring agents, emulsifiers, preservatives and the like. The compositions of the invention may be formulated so as to provide quick, sustained or delayed release of the active ingredient after their administration to a patient by employing any of the procedures well known in the art.

For example, the compositions of the present invention can be dissolved in oils, propylene glycol or other solvents which are commonly used to produce an injection. Suitable examples of the carriers include physiological saline, polyethylene glycol, ethanol, vegetable oils, isopropyl myristate, etc., but are not limited to them. For topical administration, the compounds of the present invention can be formulated in the form of ointments and creams.

Pharmaceutical formulations containing crude drug composition may be prepared in any form, such as oral dosage form (powder, tablet, capsule, soft capsule, aqueous medicine, syrup, elixirs pill, powder, sachet, granule), or topical preparation (cream, ointment, lotion, gel, balm, patch, paste, spray solution, aerosol and the like), suppository, or sterile injectable preparation (solution, suspension, emulsion).

The crude drug composition of the present invention in pharmaceutical dosage forms may be used in the form of their pharmaceutically acceptable salts, and also may be used alone or in appropriate association, as well as in combination with other pharmaceutically active compounds.

The desirable dose of the inventive composition varies depending on the condition and the weight of the subject, severity, drug form, route and period of administration, and may be chosen by those skilled in the art. However, in order to obtain desirable effects, it is generally recommended to administer at the amount ranging 0.01-10 g/kg, preferably, 1 to 5 g/kg by weight/day of the inventive composition of the present invention. The dose may be administered in a single or multiple doses per day. In terms of composition, the crude drug composition should be present between 0.01 to 80% by weight, preferably 0.5 to 50% by weight based on the total weight of the composition.

The pharmaceutical composition of present invention can be administered to a subject animal such as mammals (rat, mouse, domestic animals or human) via various routes. All modes of administration are contemplated, for example, administration can be made orally, rectally or by intravenous, intramuscular, subcutaneous, intracutaneous, intrathecal, epidural or intracerebroventricular injection.

In accordance with one aspect of the present invention, there provided a health functional food containing a wheatgrass extract for the prevention or improvement of obesity disease, as an active ingredient.

The crude drug composition of inventive health functional food is used in the form of pulverized form thereof, extracted form therefrom or dried extract form thereof.

The health functional food composition for preventing and improving purposed diseases could contain about 0.01 to 95 w/w %, preferably 0.5 to 80 w/w % of the above crude drug composition of present invention based on the total weight of the composition.

The inventive extract of the present invention also can be used as a main component or additive and aiding agent in the preparation of various functional health food and health care food.

The term “a functional health food” defined herein” the functional food having enhanced functionality such as physical functionality or physiological functionality by adding the extract of the present invention to conventional food to prevent or improve the purposed diseases in human or mammal.

It is the other object of the present invention to provide a health care food comprising a therapeutically effective amount of a wheatgrass extract, together with a sitologically acceptable additive for the prevention or alleviation of obesity disease.

The term “a health care food” defined herein “the food containing the extract or compound(s) of the present invention showing no specific intended effect but general intended effect in a small amount of quantity as a form of additive or in a whole amount of quantity as a form of beverage, gum, tea, vitamin complex, health care food, powder, granule, capsule, pill, tablet, dietary supplement etc.

The term “a sitologically acceptable additive” defined herein comprises “any substance the intended use which results or may reasonably be expected to result-directly or indirectly-in its becoming a component or otherwise affecting the characteristics of any food”, and can be classified into three groups according to its origin, i.e., (1) chemically synthetic additive such as ketones, glycin, potassium citrate, nicotinic acid, etc; (2) natural additive such as persimmon dye, licorice extract, crystalline cellulose, gua dum etc; (3) the mixed additive therewith such as sodium L-glutamate, preservatives, tar dye etc, or various categories according to its function in the food, for example, thickening agent, maturing agent, bleaching agent, sequestrant, humectant, anti-caking agent, clarifying agents, curing agent, emulsifier, stabilizer, thickener, bases and acid, foaming agents, nutrients, coloring agent, flavoring agent, sweetner, preservative agent, anti-oxidant, etc, which has been well-known in the art or previous literature (See, “Codex General Standard for Food Additives” (GSFA, Codex STAN 192-1995) in Home-page of GSFA Online: www.codexalimentarius.net/gsfaonline/index.html).

If a substance is added to a food for a specific purpose in that food, it is referred to as a direct additive and indirect food additives are those that become part of the food in trace amounts due to its packaging, storage or other handling.

The term “health care foods or health functional foods” disclosed herein can be contained in food, health beverage, dietary supplement etc, and may be formulated into a form of pharmaceutically dosing form such as a powder, granule, tablet, suspension, emulsion, syrup, chewing tablet, capsule, beverage etc; or the food form, for example, bread, rice cake, dry fruit, candy, chocolate, chewing gum, ice cream, milk such as low-fat milk, lactose-hydrolyzed milk, goat-milk, processed milk, milk product such as fermented milk, butter, concentrated milk, milk cream, butter oil, natural cheese, processed cheese, dry milk, milk serum etc, processed meat product such as hamburger, ham, sausage, bacon etc, processed egg product, fish meat product such as fish cake etc, noodle products such as instant noodles, dried noodles, wet noodles, fried noddles, non-fried noodles, gelatinized dry noodles, cooked noodles, frozen noodles, Pasta etc, tea product such as tea bag, leached tea etc, health drinks such as fruit drinks, vegetable drinks, carbonated soft drinks, soymilk drinks, lactic beverage mixed beverage, etc, seasoning food such as soy sauce, soybean paste, red pepper paste, chunjang (a kind of fermented soybean product colored by caramel), cheonggukjang (natural fermented soybean by B. subtillis), mixed paste, vinegar, sauce, ketchup, curry, dressing etc, margarine, shortening, pizza etc, but not intended herein to limit thereto, for preventing or improving of purposed disease.

Also, above described extract can be added to food or beverage for prevention and improvement of purposed disorder. The amount of above described extract or a compound(s) in food or beverage as a functional health food or health care food may generally range from about 0.01 to 100 w/w % of total weight of food for functional health food composition. In particular, although the preferable amount of the extract of the present invention in the functional health food, health care food or special nutrient food may be varied in accordance to the intended purpose of each food, it is preferably used in general to use as an additive in the amount of the extract or a compound(s) of the present invention ranging from about 0.01 to 5% in food such as noodles and the like, from 40 to 100% in health care food on the ratio of 100% of the food composition.

Providing that the health beverage composition of present invention contains above described extract or a compound(s) as an essential component in the indicated ratio, there is no particular limitation on the other liquid component, wherein the other component can be various deodorant or natural carbohydrate etc such as conventional beverage. Examples of aforementioned natural carbohydrate are monosaccharide such as glucose, fructose etc; disaccharide such as maltose, sucrose etc; conventional sugar such as dextrin, cyclodextrin; and sugar alcohol such as xylitol, and erythritol etc. As the other deodorant than aforementioned ones, natural deodorant such as taumatin, stevia extract such as levaudiosideA, glycyrrhizin et al., and synthetic deodorant such as saccharin, aspartam et al., may be useful favorably. The amount of above described natural carbohydrate is generally ranges from about 1 to 20 g, preferably 5 to 12 g in the ratio of 100 Me of present beverage composition.

The other components than aforementioned composition are various nutrients, a vitamin, a mineral or an electrolyte, synthetic flavoring agent, a coloring agent and improving agent in case of cheese, chocolate et al., pectic acid and the salt thereof, alginic acid and the salt thereof, organic acid, protective colloidal adhesive, pH controlling agent, stabilizer, a preservative, glycerin, alcohol, carbonizing agent used in carbonate beverage et al. The other component than aforementioned ones may be fruit juice for preparing natural fruit juice, fruit juice beverage and vegetable beverage, wherein the component can be used independently or in combination. The ratio of the components is not so important but is generally range from about 0 to 20 w/w % per 100 w/w % present composition. Examples of addable food comprising aforementioned extract or compound therein are various food, beverage, gum, vitamin complex, health improving food and the like.

Inventive extract or a compound(s) of the present invention has no toxicity and adverse effect therefore; they can be used with safe.

It will be apparent to those skilled in the art that various modifications and variations can be made in the compositions, use and preparations of the present invention without departing from the spirit or scope of the invention.

The present invention is more specifically explained by the following examples. However, it should be understood that the present invention is not limited to these examples in any manner.

### Advantageous Effects

As described in the present invention, the inventive composition of the present invention reduced the body weight, level of blood triglyceride, inhibited the increased level of blood insulin and leptin, reduced the level of total lipid and triglyceride in hepatic tissue, inhibited the lipid accumulation in hepatic tissue and reduced the weight of brown adipose tissue in obesity induced animal model test by high-fat diet, therefore, it can be useful in treating and preventing obesity.

### DESCRIPTION OF DRAWINGS

### Best Mode

The above and other objects, features and other advantages of the present invention will more clearly understood from the following detailed description taken in conjunction with the accompanying drawings, in which;

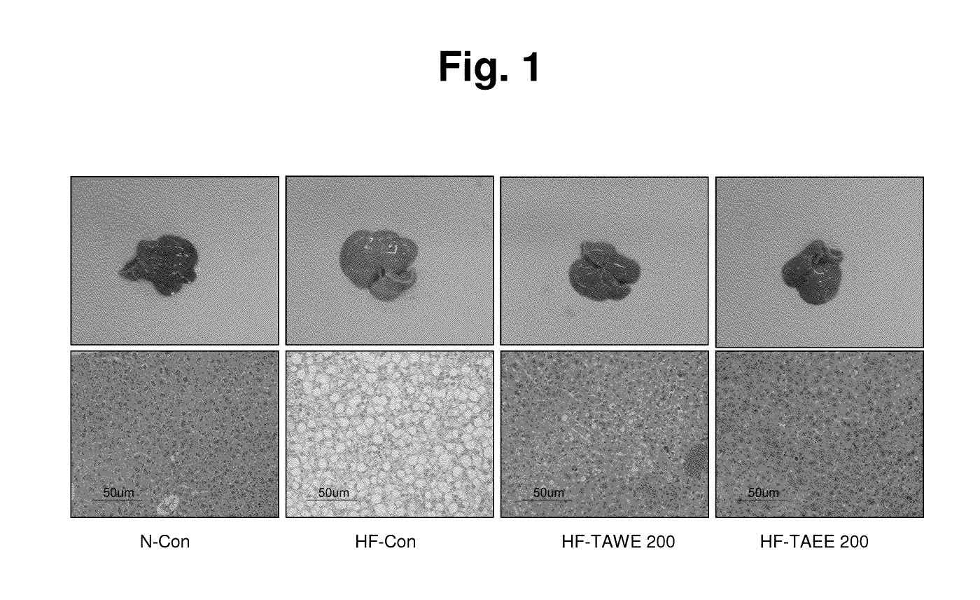

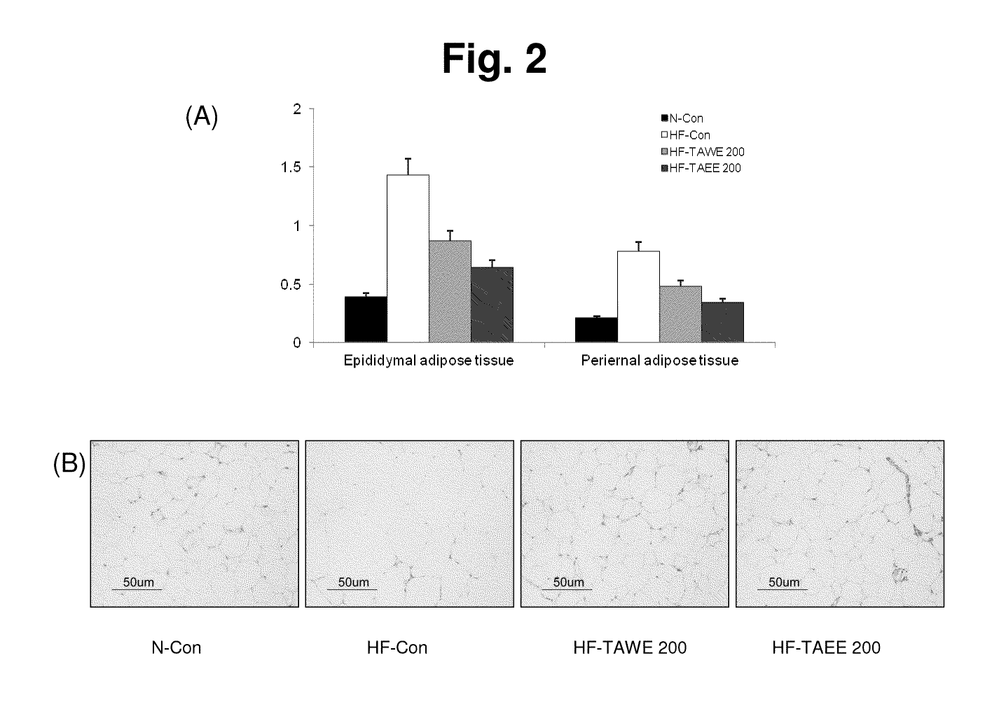

FIG. 1 shows the effect of the inventive extract on lipid accumulation in hepatic tissue stained with hematoxylin/eosin reagent and determined by histochemical examination using by mice fed with high-fat diet (ND: normal diet group, HFD: high fat diet group, HFD-TAWE: HFD+ inventive extract, HFD-TAEE: HFD+ inventive extract), FIG. 2 shows the weight of white adipose tissue (A) and the representative photographs of Epididymal adipose tissue (B) stained with H&E treated with test sample (ND: normal diet group, HFD: high fat diet group, HFD-TAWE: HFD+ inventive extract, HFD-TAEE: HFD+ inventive extract),

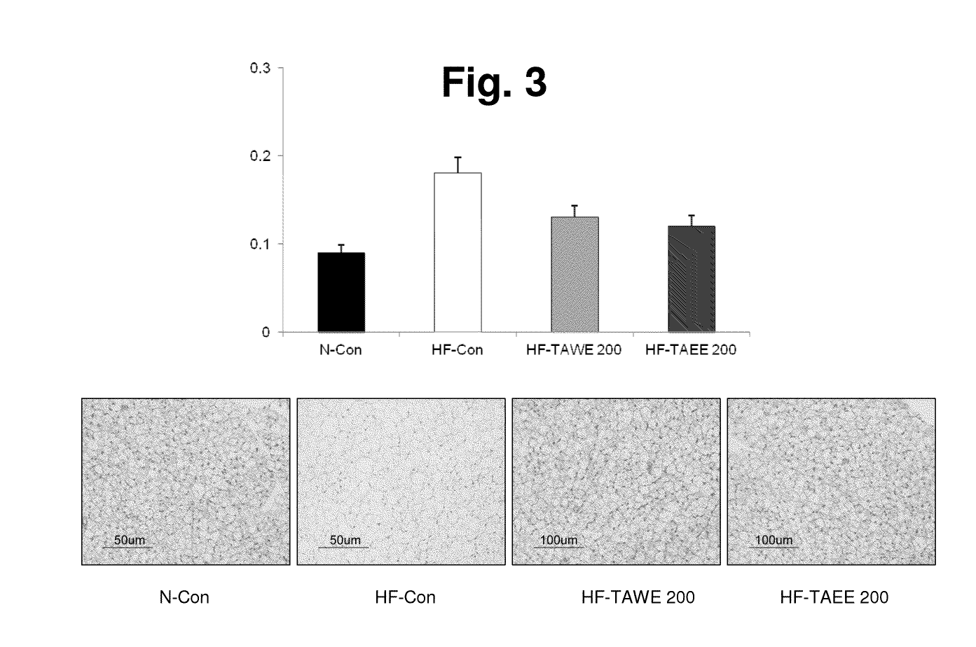

FIG. 3 represents the weight of brown adipose tissue (A) and the representative photographs of brown adipose tissue (B) stained with H&Etreated with test sample (ND: normal diet group, HFD: high fat diet group, HFD-TAWE: HFD+ inventive extract, HFD-TAEE: HFD+ inventive extract).

### BEST MODE FOR CARRYING OUT THE INVENTION

It will be apparent to those skilled in the art that various modifications and variations can be made in the compositions, use and preparations of the present invention without departing from the spirit or scope of the invention.

The present invention is more specifically explained by the following examples. However, it should be understood that the present invention is not limited to these examples in any manner.

### EXAMPLES 1

### Preparation of Each Component

### 1-1. The Wheatgrass

Triticum aestivum Lamarck purchased from NICS (National Institute of Crop Science, Korea) had been cultivated on the pear moss for sterile and organic germination at the temperature of 20±1° C. for two weeks to obtain the wheatgrass, which is further dried by freeze-drying method and used in following step.

### 1-2. The Water Extract of Wheatgrass

100 g of dried powder of wheatgrass prepared in 1-1, was added to 1 L of distilled water to extract with stirring at 90° C. for 1 hours. The extract was filtrated with Whatmann filter paper (Grade No. 1, diameter: 15 cm) and the extract was concentrated under vaccuo with rotary evaporator (EYELA Co. Ltd, Tokyo, JAPAN). The concentrate was dried with freeze-fryer (FD8512, Ilshinlab Co. Ltd, Korea) to afford the dried water extract of wheatgrass (designated as TAWE, hereinafter), which is used as a test sample in following experiment.

### 1-3. The Ethanol Extract of Wheatgrass

100 g of dried powder of wheatgrass prepared in 1-1, was added to 1 L of 95% ethanol to extract with ultra-sonic extractor (Branson 1510, USA) for 2 hours. The extract was filtrated with Whatmann filter paper (Grade No. 1, diameter: 15 cm) and the extract was concentrated under vaccuo with rotary evaporator (EYELA Co. Ltd, Tokyo, JAPAN). The concentrate was dried with freeze-fryer (FD8512, Ilshinlab Co. Ltd, Korea) to afford the dried ethanol extract of wheatgrass (designated as TAEE, hereinafter), which is used as a test sample in following experiment.

### Experimental Example 1

### Effect on Obesity Induced Animal Model

In order to investigate the inhibitory effect of the inventive extract obtained in Examples on the obesity induced animal, following experiment was performed according to the method disclosed in the literature (CHo Y. S. et al., Effect of grape seed water extract on lipid metabolism, J. Korean Soc. Food Sci. Nutr., 36(12), pp 1537-1543, 2007).

### 1-1. Experimental Animals

6-weeks aged C57BL/6J male mice (Samtako Co. Ltd., Korea) were accustomed with experimental environment for 1 week prior to the experiment and 9 mice for each experimental group were separately bred in three cages (3 mice/cage). The mice were divided into 4 groups, i.e., (a) Normal Control Group fed with normal feed (N-Con, Harlan, teklan, USA), (b) High Fat Diet Control Group fed with high-fat feed (HF-Con, AIN-76A, Research Diets Inc., D12451, ingredient: lipid 45%, 35% carbohydrate, 20% protein), (c) Test Sample Group A treated with 200 mg/kg of TAWE and fed with high-fat feed (HF+TAWE 100), (d) Test Sample Group B treated with 200 mg/kg of TAEE and fed with high-fat feed (HF+TAEE 200) and were freely accessed to feed and drinking water. The inventive extract suspended in physiological saline solution administrated into the mice at the amount of 200 mg/kg, daily for 6 weeks and only physiological saline solution was administrated into the mice according to the identical condition to the above in Normal Control Group and High Fat Diet Control Group. All animals were maintained in a controlled environment with temperatures at 20±2° C. and humidity at 50±10% with 12 hours of light and dark cycles for at least one week prior to use and all the animal model experiment was performed according to the stipulation and approval from ruled committee (Chonbuk National University Institutional Animal Care and Use Committee).

### 1-2. The Effect of Weight, Food Intake and Enemy Intake

For determining the effect of the inventive extract prepared in Example on the weight, food intake and energy intake in obesity-induced animal model, following experiment was performed.

The amount of food intake in each group was determined three times a week and the body weight of mice was determined once a week. The increased ratio of body weight (IR) in each group was determined at every predetermined time, once a week and the ratio was calculated by the following empirical formulae 1.

IR=(ET−BT)/BT [Empirical formulae 1]

wherein IR is the increased ratio of body weight,
ET is the final body weight at the end of test and
BT is the initial body weight at before test.

The energy intake was calculated by dividing the gained body weight into the amount of food intake during the same breeding period.

The inventive extract was orally administrated in a dose of 200 mg/kg, once a day, for 5 weeks and the effect of the inventive extract prepared in Example on the weight, food intake and energy intake in obesity-induced animal model was shown in Table 1.

TABLE 1
Effect on Triticum aestivum extracts on body
weight, food intake and energy intake in mice.²⁾

| | Groups¹⁾ | | | |
|---|---|---|---|---|
| Variables | ND | HFD | HFD-TAWE | HFD-TAEE |
| Initial (g) | 20.9 ± 1.26 | 20.7 ± 1.12 | 20.3 ± 0.97 | 20.5 ± 1.02 |
| final (g) | 26.6 ± 1.33a | 38.3 ± 3.41b | 34.2 ± 2.13c | 29.7 ± 2.34c |
| weight gain (g) | 5.7 ± 1.32a | 17.6 ± 2.01b | 13.7 ± 2.54c | 9.2 ± 2.26b |
| Food intake | 3.1 ± 0.53a | 4.2 ± 0.67b | 3.3 ± 0.56a | 3.0 ± 0.57a |
| (g/day) | | | | |
| Energy intake | 11.9 ± 2.94a | 22.0 ± 3.24b | 17.3 ± 3.04c | 15.1 ± 3.10c |
| (kcal/day) | | | | |

¹⁾ND, normal diet group; HFD, high fat diet group; HFD-TAWE, HFD and TAWE treatment group; HFD-TAEE, HFD and TAEE treatment group;
²⁾Values are mean ± SE (n = 7). Values with different letters in a column are significantly different at p < 0.05.

At the result, the final body weight and gained weight in normal group were 26.4±1.33 g and 5.7 g, respectively. The final body weight and gained weight in HFD group were increased by more than three folds, i.e., 38.3±3.41 g and 17.6 g, respectively whereas the gain weight in HFD-TAWE group and HFD-TAEE group was 13.7 g and 9.2 g, respectively. Comparing with the inhibitory ratio of body weight calculated by empirical formulae 2, the inhibitory ratio of body weight (%) in HFD-TAWE group and HFD-TAEE group showed 32.8% and 70.7%, respectively.

IH: inhibitory ratio of body weight (%)=100−{(GT−GN)/(GH−GN)} [Empirical formulae 2]

wherein IH is the inhibitory ratio of body weight,
GT is the gained weight in test group,
GN is the gained weight in ND, and
GH is the gained weight in HFD.

Comparing with the daily amount of food intake in each group, the daily amount of food intake in normal group and HFD group were 3.1±0.53 g/day and 4.2±0.67 g/day, respectively, while those in HFD-TAWE group and HFD-TAEE group were sharply reduced to 3.3±0.56 g/day and 2.9±0.57 g/day, respectively. In comparison with the energy intake in each group, it showed similar result to the daily amount of food intake.

Accordingly, it has been confirmed that the inventive extract has potent inhibitory effect on the increase of body weight.

### 1-3. The Effect on Blood Content of Triglyceride and Cholesterol

For determining the effect of the inventive extract prepared in Example on the blood content of cholesterol and triglyceride in obesity-induced animal model, following experiment was performed.

The obesity-induced mice were anesthetized with diethyl ether and the blood sample was collected from heart by using syringe. The collected blood was centrifuged for 20 mins at the speed of 1900×g to isolate its serum to be used as a test sample in determining the blood content level of cholesterol and triglyceride. The hepatic tissue and adipose tissue of the mice were delivered, washed with 0.9% physiological saline solution, dried with filter paper and weight of the tissues was determined.

The blood sample and tissue sample were freeze-dried and kept at −70° C. prior the use.

The blood content of triglyceride, total cholesterol and HDL cholesterol were determined by using enzymatic analysis kit {TG-S, for triglyceride (AM157) for total cholesterol (AM202), for HDL-cholesterol (AM203)} according to the manufacture's instruction manual. The blood content of triglyceride, total cholesterol and HDL cholesterol was determined at 550 nm, 550 nm and 500 nm respectively to calculate. The blood content of LDL cholesterol was calculated by Friedewald equation (Friedewald W. et al., Estimation of the concentration of low-density lipoprotein cholesterol., Clin. Chem., 18: pp. 499-502, 1972).

The effect of the inventive extract prepared in Example on the serum lipid contents in mice in obesity-induced animal model was shown in Table 2.

TABLE 2
Effect on Triticum aestivum extracts on serum lipid contents in mice.²⁾

| Factors | Groups1) | | | |
|---|---|---|---|---|
| (mg/dL) | ND | HFD | HFD-TAWE | HFD-TAEE |
| Triglyceride | 122.7 ± 12.3a | 191.0 ± 12.9b | 167.5 ± 14.2a | 142.3 ± 10.3c |
| Total cholesterol | 114.0 ± 6.6a | 151.3 ± 9.8b | 128.3 ± 7.2a | 125.9 ± 5.6a |
| HDL-cholesterol | 37.6 ± 4.2a | 28.3 ± 3.7b | 34.6 ± 4.8a | 35.7 ± 4.1a |
| LDL-cholesterol | 51.8 ± 5.7a | 84.8 ± 7.6b | 60.2 ± 8.3a | 61.7 ± 6.1a |

1)Groups are the same as in Table 1.
²⁾Values are mean ± SE (n = 7). Values with different letters in a column are significantly different at p < 0.05.

At the result, the blood content of triglyceride in normal group was 122.7±12.3 mg/dL whereas that in HFD group was increased by 55.7%, i.e., 191.0±12.9 mg/dL. However, the blood content of triglyceride in HFD-TAWE group and HFD-TAEE group was significantly reduced to 167.5±14.2 mg/dL and 142.3±10.3 mg/dL, respectively. The blood content of total cholesterol in HFD group was increased to 151.3±9.8 mg/dL while the blood content of total cholesterol in HFD-TAWE group and HFD-TAEE group was significantly reduced to 128.3±14.2 mg/dL and 125.9±11.2 mg/dL, respectively. Similarly, the blood content of LDL cholesterol in HFD group was increased while the blood content of LDL cholesterol in HFD-TAWE group and HFD-TAEE group was significantly reduced. Moreover, the blood content of HDL cholesterol in HFD group was decreased while the blood content of HDL cholesterol in HFD-TAWE group and HFD-TAEE group was significantly increased.

There have been reported that the over-consumption of saturate fatty acid and carbohydrate induces the increase of blood lipid concentration (Rim J. C. K. et al., Effect of high fat and high carbohydrate diet on serum leptin and lipids concentration in rats, Kor. J. Nutr., 34: pp. 123-131, 2001) and the occurrence ratio of hypertriglyceridemia among the Korean patients suffering with hyperlipidemia has been reported to be 17.4%, recently (Ministry of Health and Social Affairs., (2008) 2008 national health and nutrition survey: overview. Ministry of health and Welfare, Seoul, Korea. pp. 272-275).

It has been also reported that the reduce of blood LDL-cholesterol plays an important role in the treatment of patients suffering with hyperlipidemia since the increased level of total cholesterol and LDL-cholesterol has been found to be a risky factor of cardiovascular disease (National Cholesterol Education Program Expert Panel., (2002) Third Report of the National Cholesterol Education Program Expert Panel on Detection, Evaluation and Treatment of High Blood Cholesterol in Adults (Adult Treatment Panel III) final report. Circulation 106: pp. 3143-3421).

### 1-4. The Effect on Blood Content of Glucose, Leptin and Insulin

For determining the effect of the inventive extract prepared in Example on the blood content of glucose, leptin and insulin in obesity-induced animal model, following experiment was performed.

The obesity-induced mice were anesthetized with diethyl ether and the blood sample in obesity-induced mice was collected from the caudal vena cava of the mice at pre-determined time, for every week. The collected blood was left alone for 1 hour and centrifuged at 4° C. at the speed of 3000 rpm to isolate its plasma to be used as a test sample in determining the blood content level of glucose, leptin and insulin. The content of insulin in the blood was determined by using blood glucose monitor (Abbott Diabetes Care Ltd.). The level of insulin in the blood was determined by using ELISA kit (Shibayagi Co., JAPAN) according to the manufacture's instruction manual.

To confirm the inhibitory activity of inventive extract on the level of released insulin and leptin in high-fat diet mice, the inventive extract was orally administrated for 5 weeks and at the end of the test, the level of insulin and leptin was determined.

The determined result of blood insulin and leptin was shown in Table 3.

TABLE 3
Effect on TAWE and TAEE on insulin and leptin in the blood.²⁾

| | Groups | | | |
|---|---|---|---|---|
| Variables | ND | HFD | HFD-TAWE 200 | HFD-TAEE 200 |
| Insulin (ng/ml) | 2.33 ± 0.57 | 4.16 ± 1.35 | 3.17 ± 0.97 | 2.86 ± 1.13 |
| Leptin (ng/ml) | 2.16 ± 1.21 | 29.55 ± 6.79 | 11.23 ± 3.56 | 7.98 ± 3.26 |

Data are mean ± SE values of 7 mice per group. Values in the same row not sharing common superscript letters are significantly different at p < 0.05, as assessed using Ducan's multiple range test.
N-Con, normal diet group; HF-Con, high fat diet group; HF-TAWE 200; HF-TAEE 200 group.

At the result, the blood level of leptin and insulin in HFD group was increased by 13.6 fold and 1.28 fold, respectively, i.e., 29.55±6.79 ng/ml and 4.16±1.35 ng/ml, compared with normal control group (ND). However, the blood level of leptin and insulin in HFD-TAWE 200 group was slightly increased by only 5.1 fold and 1.36 fold, respectively, and those in HFD-TAEE 200 group was slightly increased by only 3.19 fold and 1.22 fold, respectively.

There have been reported that the main cause of the insulin resistance in beta cell is the glucose toxicity of hyperglycemia and lipotoxicity resulting transformed triglyceride (Hanefeld M. et al., Impaired fasting glucose is not a risk factor for atherosclerosis, Diabet Med., 16, p. 212, 1999). Accordingly, it has been confirmed that the inventive extract showed potent treating effect on insulin resistance and improving effect on insulin release in type-II diabetes.

### 1-5. The Effect on Hepatic Tissue and Adipose Tissue

For determining the effect of the inventive extract prepared in Example on the hepatic tissue and adipose tissue in obesity-induced animal model, following experiment was performed. The obesity-induced mice were anesthetized with diethyl ether and for histological examination, the hepatic tissue, epididymal adipose tissue and perirenal adipose tissue in obesity-induced mice were delivered and the weight of each tissue was determined. The tissue was fixed in 10% formalin solution and washed with PBS. The paraffin block was prepared and sliced into tissue slices with the width of 5 μm using by microtome. The paraffin in the tissue slice was removed with xylene through deparaffination process and the nucleus and cytoplasm of the tissue was stained for 5 mins with H&E (Sigma Co. Ltd.) to observe the morphology of the tissue in each group using by optical microscopy (200×).

In order to confirm the inhibitory activity of inventive extract on the increase of lipid accumulation in hepatic tissue of high fat diet mice, the weight of liver and the content of total lipid and neutral lipid in hepatic tissue were analyzed. The result was shown in Table 4.

TABLE 4
Effect of Triticum aestivum extracts on liver
weights and lipid contents in high fat-fed mice²⁾

| | Groups¹⁾ | | | |
|---|---|---|---|---|
| Factors | ND | HFD | HFD-TAWE | HDF-TAEE |
| Liver weight (g) | 1.20 ± 0.34^{a} | 2.65 ± 0.45^{b} | 1.64 ± 0.21^{a} | 1.31 ± 0.34^{a} |
| Total lipid (mg/g) | 62.14 ± 3.14^{a} | 178.21 ± 12.07^{b} | 79.35 ± 4.45^{a} | 68.34 ± 3.14^{a} |
| Triglyceride (mg/g) | 31.45 ± 2.10^{a} | 67.66 ± 3.11^{b} | 41.32 ± 2.20^{a} | 34.14 ± 2.65^{a} |

¹⁾Groups are the same as in Table 1.
²⁾Values are mean ± SE (n = 7). Values with different letters in a column are significantly different at p < 0.05.

The weight of liver in normal control group was 1.20±0.34 g whereas that in HFD group was increased to 2.65±0.45 g. However, the weight of liver in HFD-TAWE group and in HFD-TAEE group was reduced by 38% and 50% compared with HFD group. The content of total lipid and neutral lipid within the hepatic tissue in HFD group was increased by 186.7% and 115.1% compared with normal control group. The content of total lipid and neutral lipid within the hepatic tissue in HFD-TAWE group was increased by only 27.7% and 31.4% compared with normal control group and the content of total lipid and neutral lipid within the hepatic tissue in HFD-TAEE group was increased by only 9.9% and 8.5% compared with normal control group. Accordingly, it has been confirmed that the inventive extract, TAWE as well as TAEE, showed potent lowering effect on the content of total lipid and neutral lipid within the hepatic tissue.

The increased size and weight of liver accompanying by high fat diet is caused by the lipid accumulation of hepatic tissue and therefore, the present inventors prepared the paraffinized tissue slices and consecutively stained the tissue with Hematoxylin (Sigma co. Ltd. MHS-16) and Eosin (Sigma co. Ltd. HT110-1).

As can be seen in FIG. 1, the morphology of delivered liver tissue in Normal control group showed dark scarlet color overall without lipid vesicle while that in HFD group showed light pink color deposited with yellow or white colored lipid droplet and lots of hepatic intracellular lipid vesicles overall and increased size and weight of liver. However, the morphology of delivered liver tissue in HFD-TAWE group showed remarkably reduced lipid accumulation with partly deposited lipid vesicle and that in HFD-TAEE group showed clearly reduced lipid accumulation, similarly to that in normal control group.

In order to confirm the inhibitory effect of inventive extract on the density of white adipose tissue having a long-term energy-storage function and brown adipose tissue having an energy-consuming function in the body, the inventive extract with high fat diet was orally administrated into the mice for 5 weeks and for histological examination, the epididymal adipose tissue and the perirenal adipose tissue in the mice, were delivered. The weight of each tissue was determined and the tissue was stained with H&E to observe the morphology of the epididymal adipose tissue and the perirenal adipose tissue by optical microscopy.

As can be seen in FIG. 2, the weight of white adipose tissue in epididymal adipose tissue in HFD group was increased by 3.6 folds, 1.43±0.55 g, compared with that in normal control group (0.39±0.09 g). However, the weight of epididymal adipose tissue in HFD-TAWE group and HFD-TAEE group were sharply reduced by 49% (0.87±0.18 g) and 55% (0.64±0.14 g), respectively, compared with that in HFD group and the weight of perirenal adipose tissue in HFD-TAWE group and HFD-TAEE group were sharply reduced by 38% and 56%, respectively, compared with that in HFD group. Accordingly, it has been confirmed that the inventive extract if the present invention showed potent lowering effect on the increased white adipose tissue.

As can be seen in FIG. 3, the weight of brown adipose tissue in perirenal adipose tissue in HFD group, normal control group, HFD-TAWE group and HFD-TAEE group was 0.18±0.098 g, 0.09±0.057 g, 0.13±0.081 g and 0.12±0.073 g, respectively, Accordingly, it has been confirmed that the inventive extract if the present invention showed potent lowering effect on the increased brown adipose tissue.

There have been reported that the increase of body fat, in particular, the increase of abdominal adipose tissue rather than increase of body weight, has been as a generator of risk factors for cardiovascular disease and diabetes (Bjorntorp P., The associations between obesity, adipose tissue distribution and disease. Acta Med Scand., 723: pp. 121-134, 1998; Bjorntorp P., “Portal” adipose tissue as a generator of risk factors for cardiovascular disease and diabetes. Arteriosclerosis, 10: pp. 493-496, 1990)

### 1-6. Stastistics

All the results was expressed as mean±S.D (Standard deviation) and analyzed by using SPSS (statistical package for social science). The statistic significance for mean difference between each group was analyzed by ANOVA and verified at p<0.05 using by Duncan's multiple range test.

### MODE FOR INVENTION

Hereinafter, the formulating methods and kinds of excipients will be described, but the present invention is not limited to them. The representative preparation examples were described as follows.

### Preparation of Infection

| | |
|---|---|
| TAEE | 200 mg |
| Mannitol | 180 mg |
| Na₂HPO₄, 12H₂O | 26 mg |
| Distilled water for injection | 2974 mg |

Injection preparation was prepared by dissolving active component, controlling pH to about 7.5 and then filling all the components in 2 Ml ample and sterilizing by conventional injection preparation method.

### Preparation of Powder

| | |
|---|---|
| TAEE | 200 mg |
| Lactose | 100 mg |
| Talc | 10 mg |

Powder preparation was prepared by mixing above components and filling sealed package.

### Preparation of Tablet

| | |
|---|---|
| TAEE | 200 mg |
| Corn Starch | 100 mg |
| Lactose | 100 mg |

Magnesium stearate 2 mg

Tablet preparation was prepared by mixing above components and entabletting.

### Preparation of Capsule

| | | |
|---|---|---|
| TAEE | 200 | mg |
| Lactose | 14.8 | mg |
| Microcrystalline celluose | 3 | mg |
| Magnesium stearate | 0.2 | mg |

Capsule preparation was prepared by mixing above components and filling gelatin capsule by conventional gelatin preparation method.

### Preparation of Liquid

| | | |
|---|---|---|
| TAEE | 200 | mg |
| Isomerose | 10 | g |
| Mannitol | 5 | g |

| | |
|---|---|
| Distilled water | optimum amount |

Liquid preparation was prepared by dissolving active component, and then filling all the components in 100 Ml ample and sterilizing by conventional liquid preparation method.

### Preparation of Health Food

| TAEE | 1000 | mg |
|---|---|---|
| Vitamin mixture | optimum amount | |
| Vitamin A acetate | 70 | μg |
| Vitamin E | 1.0 | mg |
| Vitamin B₁ | 0.13 | mg |
| Vitamin B₂ | 0.15 | mg |
| Vitamin B₆ | 0.5 | mg |
| Vitamin B₁₂ | 0.2 | μg |
| Vitamin C | 10 | mg |
| Biotin | 10 | μg |
| Amide nicotinic acid | 1.7 | mg |
| Folic acid | 50 | μg |
| Calcium pantothenic acid | 0.5 | mg |
| Mineral mixture | optimum amount | |
| Ferrous sulfate | 1.75 | mg |
| Zinc oxide | 0.82 | mg |
| Magnesium carbonate | 25.3 | mg |
| Monopotassium phosphate | 15 | mg |
| Dicalcium phosphate | 55 | mg |
| Potassium citrate | 90 | mg |
| Calcium carbonate | 100 | mg |
| Magnesium chloride | 24.8 | mg |

The above mentioned vitamin and mineral mixture may be varied in many ways. Such variations are not to be regarded as a departure from the spirit and scope of the present invention.

### Preparation of Health Beverage

| | | |
|---|---|---|
| TAEE | 1000 | mg |
| Citric acid | 1000 | mg |
| Oligosaccharide | 100 | g |
| Apricot concentration | 2 | g |
| Taurine | 1 | g |
| Distilled water | 900 | Ml |

Health beverage preparation was prepared by dissolving active component, mixing, stirred at 85° C. for 1 hour, filtered and then filling all the components in 2000 Ml ample and sterilizing by conventional health beverage preparation method.

The invention being thus described, it will be obvious that the same may be varied in many ways. Such variations are not to be regarded as a departure from the spirit and scope of the present invention, and all such modifications as would be obvious to one skilled in the art are intended to be included within the scope of the following claims.

### INDUSTRIAL APPLICABILITY

As described in the present invention, the inventive extract reduced the body weight, level of blood triglyceride, inhibited the increased level of blood insulin and leptin, reduced the level of total lipid and triglyceride in hepatic tissue, inhibited the lipid accumulation in hepatic tissue and reduced the weight of brown adipose tissue in obesity induced animal model test by high-fat diet, therefore, it can be useful in treating and preventing obesity.

1. A pharmaceutical composition containing a wheatgrass extract for the prevention and treatment of obesity disease.

2. The pharmaceutical composition of claim 1, wherein said wheatgrass extract is obtained by extracting the sprout with water, C1-C4 lower alcohols or the mixtures thereof.

3. The pharmaceutical composition of claim 1, wherein said wheatgrass extract is obtained by extracting the sprout with water or the mixtures of water and ethanol.

4. The pharmaceutical composition of claim 1, wherein said wheatgrass is the sprout of Triticum genus plants such as Triticum aestivum Lamarck, Triticum vulgare, Triticum durum, Triticum compactum and the like, having been cultivated from germination for the period from 1 week to 7 weeks.

5. A health functional food containing a wheatgrass extract for the prevention or improvement of obesity disease, as an active ingredient.

6. The health functional food of claim 5, wherein said health functional food is provided as a form of beverage, gum, tea, vitamin complex, health care food, powder, granule, capsule, pill, tablet, dietary supplement etc.

7. A health care food comprising a therapeutically effective amount of a wheatgrass extract, together with a sitologically acceptable additive for the prevention or alleviation of obesity disease.

8. A method for treating obesity disease in a mammal comprising administering to said mammal an effective amount of a wheatgrass extract, together with a pharmaceutically acceptable carrier thereof.

9. A use of a wheatgrass extract for manufacture of medicament employed for treating or preventing obesity disease in human or mammal.
